(19) United States
(12) Patent Application Publication
(10) Pub. No.: US 2017/0065810 A1
(43) Pub. Date: Mar. 9, 2017

(54) INTEGRATED VALVE ASSEMBLY

(71) Applicant: AptarGroup, Inc., Crystal Lake, IL (US)

(72) Inventors: John M. Hess, Midland, MI (US); Roger H. Falde, Mahtomedi, MN (US); James P. Manning, Sanford, MI (US); John D. Hall, Grand Rapids, MI (US); Mark R. Honard, Saginaw, MI (US)

(73) Assignee: AptarGroup, Inc., Crystal Lake, IL (US)

(21) Appl. No.: 15/122,705

(22) PCT Filed: May 14, 2014
(86) PCT No.: PCT/US14/00105

§ 371 Date: Aug. 31, 2016

(51) Int. Cl. A61M 39/24

(52) U.S. Cl. CPC A61M 39/24; A61M 2039/242; A61M 2039/2426

(57) ABSTRACT

An integrated valve assembly (10, 110, 210, 310, 410) is provided for pressure-responsive flow control for associated components, such as for nutritional administration to patients. The valve assembly includes a first connector element (12, 112, 212, 312, 412) and a second connector element (14, 114, 214, 314, 414), which are joined to each other, and which respectively define a first, internal flow passage (20, 120, 220, 320, 420) and a second internal flow passage (28, 128, 228, 328, 428). A valve member (40, 140, 240, 340, 440), which may comprise a resiliently deformable slit valve, is positioned between the first connector element (12, 112, 212, 312, 412) and the second connector element (14, 114, 214, 314, 414) for providing pressure-responsive flow control between the first, internal flow passage (20, 120, 220, 320, 420), and the second, internal flow passage (28, 128, 228, 328, 428). At least one of the first connector element (12, 112, 212, 312, 412) and the second connector element (14, 114, 214, 314, 414) can be configured in accordance with ISO CD 80369-3 for use in enteral nutritional administration.

### CROSS-REFERENCE TO RELATED APPLICATIONS

Not Applicable.

### FEDERALLY SPONSORED RESEARCH OR DEVELOPMENT

Not Applicable.

### MICROFICHE/COPYRIGHT REFERENCE

Not Applicable.

### FIELD OF THE INVENTION

The present invention relates generally to an integrated valve assembly which can be advantageously used for patient care and like medical applications, and more particularly to an integrated valve assembly which can be configured to include connection portions adapted for specific medical applications, and flow-control characteristics consistent with such applications.

### BACKGROUND OF THE INVENTION

Many medical applications require flow-controlling devices for controlling flow of products for enteral nutritional administration, intravenous administration including parenteral nutritional administration, and like patient care. To promote efficient use for various medical applications, systems and devices for administering solutions and other compounds can be configured to facilitate use for certain applications, while precluding use for other applications. For example, various types of tubing connectors and the like can be configured for enteral nutritional administration, while the same connectors cannot be properly used for other applications, such as intravenous administration.

Additionally, certain medical applications require certain flow characteristics, including pressure-responsive flow control, as well as reverse-flow prevention.

The present invention contemplates an integrated valve assembly which can be specifically configured for specific medical applications, and can also be configured to exhibit flow-control characteristics as may be required by the specific application.

### SUMMARY OF THE INVENTION

In accordance with one feature of the invention, an integrated valve assembly is provided for controlling flow of flowable substances, which assembly is particularly suitable for medical applications, and can be configured for enteral nutritional administration. To this end, the valve assembly can include connection portions which are specifically configured for a selected type of medical application, with these connection portions typically being suited for use for that application only. This desirably avoids inadvertent connection of components associated with one type of patient care, e.g. enteral nutritional administration, with another type of patient care, e.g. intravenous administration.

In one feature, the flow-control characteristics of the valve assembly can be specifically configured for the desired application, including anti-free flow characteristics, anti-reflux characteristics, and anti-backflow characteristics.

As one feature, in accordance with the illustrated embodiments, the present integrated valve assembly comprises a first connector element having a first connection portion at one end thereof, and a first annular valve interface at an opposite end thereof. The first connector element defines a first, internal flow passage.

According to one feature, the present valve assembly further includes a second connector element having a second connection portion at one end thereof and a second annular valve interface at an opposite end thereof. The first and second connector elements are joined to each other with the first and second valve interfaces arranged in confronting relationship with each other.

As one feature, the present valve assembly further includes a resiliently deformable valve member positioned between the first and second connector elements at the first and second valve interfaces. The valve member controls flow between the first and second internal passages of the first and second connector elements for controlling flow through the valve assembly.

In one feature, the configuration of the valve member can be varied, depending upon the specific application of the valve assembly. The valve member may be a bi-directional valve, or a one-way valve, with the arrangement providing for controlled flow of powders, gels, gas, liquids, and fluids. For enteral nutritional administration, the valve member can be configured to provide anti-free flow, anti-reflux or anti-backflow characteristics, such as during nutritional administration, prior, during, or after infusion.

A resiliently deformable valve member suitable for use in the present integrated valve assembly can be configured in accordance with the teachings of U.S. Pat. No. 5,839,614, hereby incorporated by reference. Other designs or types of valves (not illustrated) may be employed instead.

In a further feature, for enteral nutritional administration, at least one of the first and second connection portions of the first and second connectors can be configured in accordance with ISO CD 80369-3. For other applications, such as for intravenous or dialysis administration, the connection portion of one or both of the connections can be varied, as may be required for that application.

As a further feature, depending upon the specific application, the valve member of the valve assembly can be configured to prevent flow in a first direction through the valve assembly below a predetermined pressure differential. The valve member can be configured to permit flow in a first direction through the valve assembly, while preventing reverse flow (at least below a predetermined pressure differential) in a second direction, opposite the first direction, through the valve assembly.

In yet another feature, for nutritional administration, the present valve assembly can be specifically configured depending upon the specific use in a nutritional administration system. For example, when configured for anti-free flow control, the present valve assembly can be arranged to prevent free flow of the liquid from an associated nutritional solution container through tubing to a patient, the administration of which is ordinarily controlled by a peristaltic pump. For such an application, it is desirable to configure the valve assembly such that the valve assembly remains closed at pressures below 96 inches of water, as may be created by the static head of a suspended container of nutritional solution for example, with the valve assembly opening at pressures greater than 130 inches of water, as may be created by operation of the peristaltic pump. Such an arrangement prevents inadvertent flow in the event that the pump becomes disengaged from the administration system and the tubing between the suspended container and the patient is not otherwise manually closed (e.g., by a roller clamp or the like).

According to one feature, when configured for anti-reflux use, the present valve assembly can be operatively connected with the J-tube provided for a patient, thus acting to preclude undesired reflux from the patient, as can occur if the patient is moved, coughs, or the like.

In one feature, given the manner in which the flow characteristics of the present valve can be specifically tailored for the desired specific application, it can sometimes be desirable to use plural ones of the valve assemblies in series with each other, including connection of one valve assembly to another.

It should be appreciated that the invention may include any or all of the above described features, include only one of the above features, more than one of the above features, and any combination of the above features. Furthermore, other objects, features, and advantages of the invention will become apparent from a review of the entire specification, including the drawings.

In one feature, the first connector portion of the first connector element comprises a male threaded connector configured for enteral nutritional administration in accordance with ISO CD 80369-3, and the second connector portion of the second connector of the second connector element comprises a female threaded connector configured for enteral nutritional administration in accordance with ISO CD 80369-3. The valve member comprises a resiliently deformable slit valve configured to permit flow in a first direction to the valve assembly above a predetermined pressure differential acting in a first direction, and to prevent reverse flow in a second direction, opposite the first direction, through the valve assembly below a predetermined pressure differential acting in a second direction.

According to one feature, the first connector portion of the first connector element comprises a male threaded connector configured for enteral nutritional administration in accordance with ISO CD 80369-3, and the second connector portion of the second connector element comprises an annular undercut connector. The valve member comprises a resiliently deformable slit valve configured to permit flow in a first direction through the valve assembly above a predetermined pressure differential acting in the first direction, and to prevent reverse flow in a second direction, opposite the first direction through the valve assembly below a predetermined pressure differential acting in a second direction.

In a further feature, the first connector portion of the first connector element comprises a female threaded connector configured for enteral nutritional administration in accordance with ISO CD 80369-3, and the second connector portion of the second connector element comprises an annular undercut connector. The valve member comprises a resiliently deformable slit valve configured to permit flow in a first direction through the valve assembly above a predetermined pressure differential acting in the first direction, and to prevent reverse flow in a second direction, opposite the first direction, through the valve assembly below a predetermined pressure differential acting in a second direction.

As a further feature, the first connector portion of the first connector element comprises a tubular connector, and the second connector portion of the second connector element comprises a female threaded connector configured for enteral nutritional administration in accordance with ISO CD 80369-3. The valve member comprises a resiliently deformable slit valve configured to permit flow in a first direction through the valve assembly above a predetermined pressure differential acting in the first direction, and to prevent reverse flow in a second direction, opposite the first direction, through the valve assembly below a predetermined pressure differential acting in the second direction. The second annular valve interface of the second connector element includes a second, annular valve seat, and a second annular attachment flange positioned radially outwardly of the second annular valve seat, wherein the first and second annular attachment flanges are joined to each other.

In one feature, the valve member is a resiliently deformable slit valve comprising a head portion defining at least one slit, a peripheral attachment portion positioned between said first annular valve seat and said second seat, and a sleeve portion extending between and joining said head portion and said peripheral attachment portion.

According to one feature, the integrated valve assembly comprises a first connector element having a first connection portion at one end thereof, and a first annular valve interface at an opposite end thereof, wherein the first connector element defines a first, internal flow passage. A second connector element has a second connection portion at one end thereof, and a second annular valve interface at an opposite end thereof, wherein the first and second connector elements are joined to each other with the first and second valve interfaces arranged in confronting relationship with each other.

As a further feature, the first annular valve interface of the first connector element includes a first, annular valve seat, and a first annular attachment flange positioned radially outwardly of the first annular valve seat, with the second annular valve interface of the second connector element including a second annular valve seat, and a second annular attachment flange positioned radially outwardly of the second annular valve seat, wherein the first and second annular attachment flanges are joined to each other. At least one of the first connection portion of said first connector and said second connection portion of said second connector is configured for enteral nutritional administration in accordance with ISO CD 80369-3.

In another feature, a valve member positioned between the first and second connector elements at the first and second valve interfaces is provided, wherein the valve member controls flow between the first and second internal flow passages of the first and second connector elements for controlling flow through the valve assembly, wherein the valve member comprises a resiliently deformable slit valve configured to permit flow in a first direction through the valve assembly above a predetermined pressure differential acting in the first direction, and to prevent reverse flow in a second direction, opposite the first direction, through the valve assembly below a predetermined pressure differential acting in the second direction, wherein the valve member comprises a head portion defining at least one slit, a peripheral attachment portion positioned between said first, annular valve seat and second, annular seat, and a sleeve portion extending between and joining said head portion and said peripheral attachment portion.

Numerous other advantages and features of the present invention will become readily apparent from the following detailed description of the invention, from the claims, and from the accompanying drawings.

### BRIEF DESCRIPTION OF THE DRAWINGS

In the accompanying drawings forming part of the specification, in which like numerals are employed to designate light parts throughout the same.

FIG. 1 is an isometric view taken from an exterior side of an integrated valve assembly embodying the present invention;

FIG. 2 is a cross-sectional view of the integrated valve assembly shown in FIG. 1;

FIG. 3 is an isometric view from an exterior side of a connector element of the integrated valve assembly shown in FIG. 1;

FIG. 4 is a cross-sectional view of the connector element shown in FIG. 3 of the present integrated valve assembly;

FIG. 5 is an isometric view from an exterior side of a connector element of the integrated valve assembly shown in FIG. 1;

FIG. 6 is a cross-sectional view of the connector element shown in FIG. 5;

FIG. 7 is an isometric view from an exterior side of a valve member of the integrated valve assembly shown in FIG. 1;

FIG. 8 is a cross-sectional view of the valve member shown in FIG. 7;

FIG. 9 is an isometric view from an exterior side of an alternate embodiment of the present integrated valve assembly;

FIG. 10 is a cross-sectional view of the integrated valve assembly shown in FIG. 9;

FIG. 11 is an isometric view of an exterior side of a connector element of the integrated valve assembly shown in FIG. 9;

FIG. 12 is a cross-sectional view of the connector element shown in FIG. 11;

FIG. 13 is an isometric view of an exterior side of a further alternate embodiment of the present integrated valve assembly;

FIG. 14 is a cross-sectional view of the integrated assembly shown in FIG. 13;

FIG. 15 is an isometric view from an exterior side of a further alternate embodiment of the present integrated valve assembly;

FIG. 16 is a cross-sectional view of the integrated valve assembly shown in FIG. 15;

FIG. 17 is an isometric view of an exterior side of a connector element of the integrated valve assembly as shown in FIG. 15;

FIG. 18 is a cross-sectional view of the connector element shown in FIG. 17;

FIG. 19 is an isometric view of an exterior side of a further alternate embodiment of the present integrated valve assembly; and

FIG. 20 is a cross-sectional view of the integrated valve assembly shown in FIG. 19.

### DETAILED DESCRIPTION OF THE PREFERRED EMBODIMENTS

While this invention is susceptible of embodiments in many different forms, this specification and the accompanying drawings disclose specific forms as examples of the invention. The invention is not intended to be limited to the embodiments so described, however. The scope of the invention is pointed out in the appended claims.

As discussed in detail hereinafter, many medical applications require flow-controlling devices for controlling flow of products for enteral nutritional administration, intravenous administration including parenteral nutritional administration, and like patient care. To promote efficient use for various medical applications, systems and devices for administering solutions and other compounds can be configured to facilitate use for certain applications, while precluding use of other applications. For example, various types of tubing connectors and the like can be configured for enteral nutritional administration, while the same connectors cannot be properly used for other applications, such as intravenous administration.

Additionally, certain medical applications require certain flow characteristics, including pressure-responsive flow control, as well as reverse-flow prevention.

The present invention contemplates an integrated valve assembly which can be specifically configured for specific medical applications, and can also be configured to exhibit flow-control characteristics as may be required for this specific application. In accordance with the illustrated embodiments, the present integrated valve assembly has been particularly configured for enteral nutritional administration, and to this end, includes one or more connector elements configured in accordance with ISO CD 80369-3. For other application, such as for intravenous or dialysis administration, the specific configuration of the connector elements can be varied, as may be required for that application. For some applications, the connector elements can be configured as a so-called cassette for providing flow control for liquid through associated tubing joined to the cassette.

As will be further described in detail, the present integrated valve assembly comprises a first connector element having a first connector portion at one thereof, and a first annular valve interface at an opposite end thereof. The first connector element defines a first, internal flow passage.

The present valve assembly further includes a second connector element having a second connection portion at one thereof and a second annular valve interface at an opposite end thereof. The second connector element defines a second, internal flow passage. The first and second connector elements are joined to each other with the first and second valve interfaces arranged in confronting relationship with each other.

In order to control flow through the valve assembly, the assembly includes a resiliently deformable valve member positioned between the first and second connector elements at the first and second valve interfaces. The valve member controls flow between the first and second internal passages of the first and second connector elements, thereby controlling flow through the valve assembly.

The configuration of the valve member can be varied, depending upon the specific application of the valve assembly. The valve member may be a bi-directional valve, or a one-way valve, with the arrangement providing for control flow of powders, gels, gas, liquids, and fluids. For enteral nutritional administration, the valve member can be configured to provide anti-free flow, anti-reflex or anti-backflow characteristics, such as during nutritional administration, prior, during, or after infusion.

Depending upon the specific application, the valve member of the valve assembly can be configured to prevent flow in a first direction through the valve assembly below a predetermined pressure differential. The valve member can be configured to permit flow in a first direction through the valve assembly, while preventing reverse flow, at least below a predetermined pressure differential, in a second direction, opposite the first direction, through the valve assembly. For nutritional administration, the present valve assembly can be specifically configured depending upon the specific use in a nutritional administration system. For example, when configured for anti-free flow control, the present valve assembly can be arranged to prevent free flow of liquid from an associated nutritional solution container through tubing to a patient, the administration of which is ordinarily controlled by a peristaltic pump. For such an application it is desirable to configure the valve assembly such that the valve assembly remains closed at pressures below 96 inches of water, as may be created by the static head of a suspended container of nutritional solution for example, with the valve assembly opening at pressures greater than 130 inches of water, as may be created by operation of peristaltic pump. Such an arrangement prevents inadvertent flow in the event that the pump becomes disengaged from the administration system and the tubing between the suspended container and the patient is not otherwise manually enclosed, such as by a suitable roller clamp or the like.

When configured for anti-reflux use, the present valve assembly can be operatively connected with the J-tube provided for a patient, thus acting to preclude undesired reflux from the patient, as can occur if the patient moves, coughs, or the like. Given the manner in which the flow characteristics of the present valve assembly can be specifically tailored for the desired specific application, it can sometimes be desirable to use plural ones of the valve assemblies in series with each other including connection with one valve assembly to another.

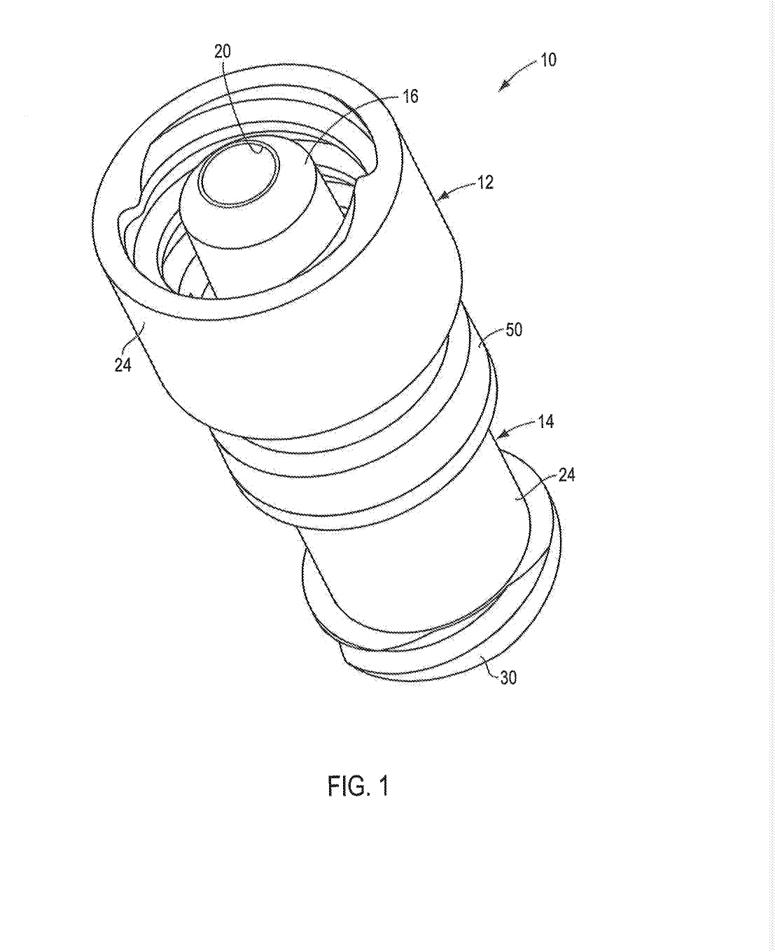

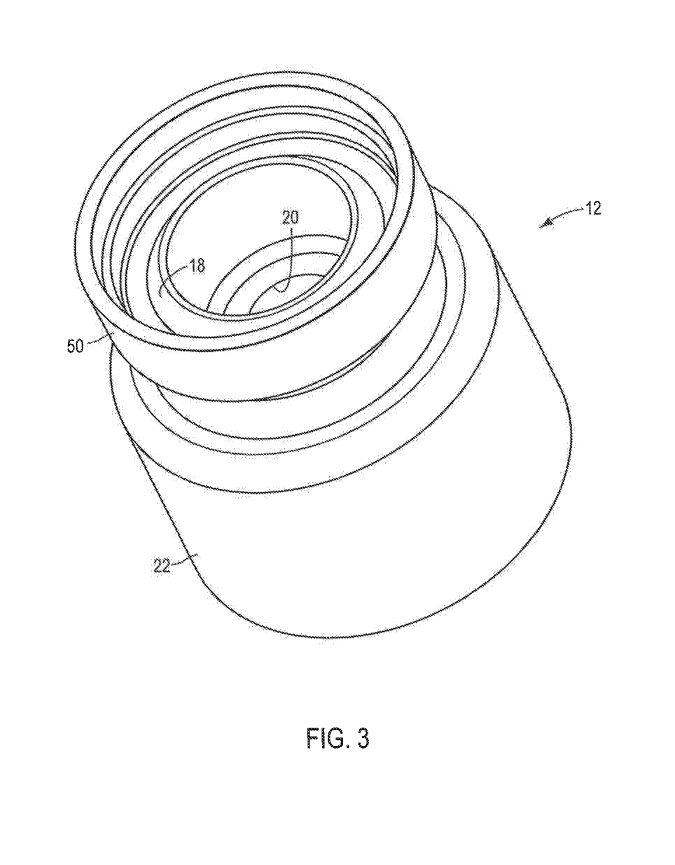

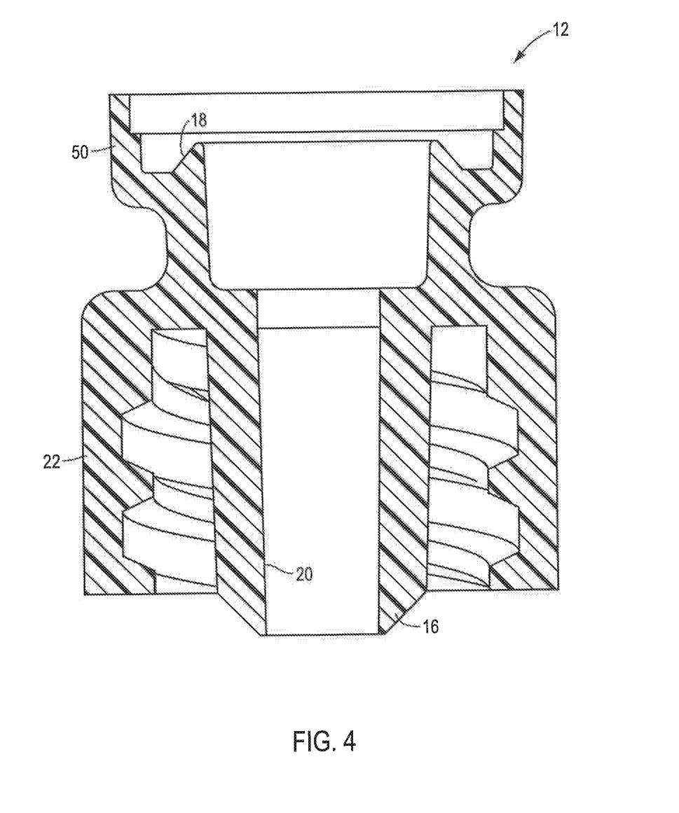

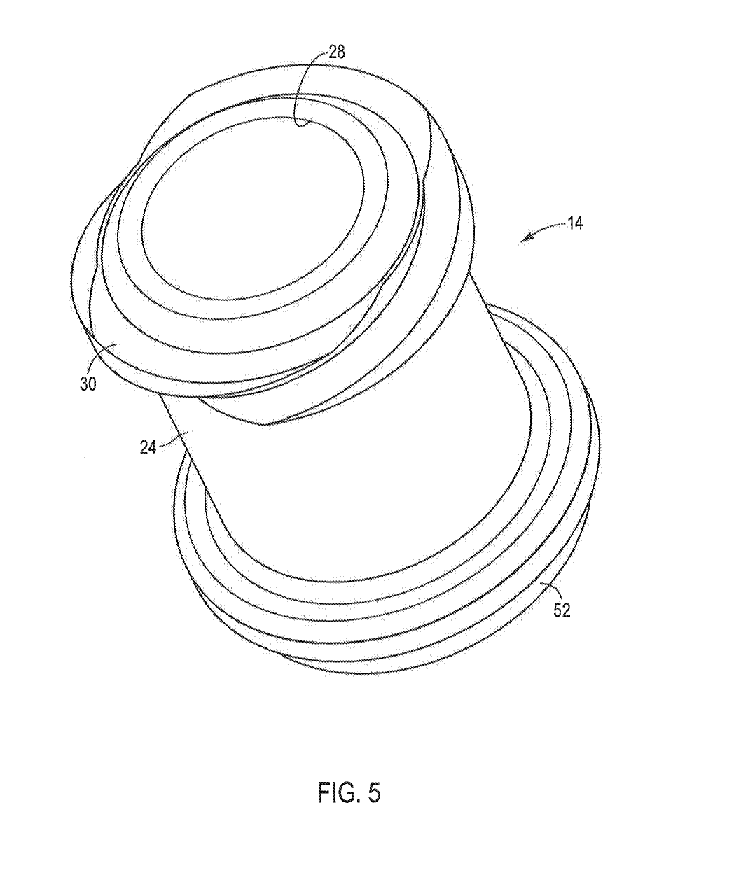

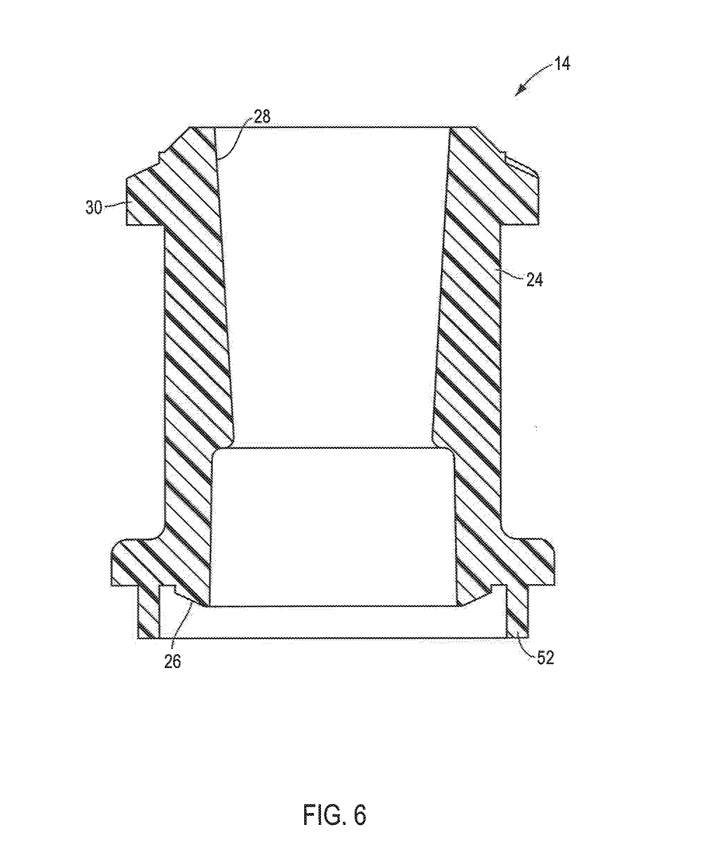

With reference now to FIGS. 1-8, therein is illustrated a first preferred embodiment of the present integrated valve assembly of the present invention, designated 10. While integrated valve assembly 10 can be configured for a wide variety of applications where pressure-responsive liquid flow is desired, this embodiment of the integrated valve assembly, like the embodiments described hereinafter, has been particularly configured for enteral nutritional administration, with components of the assembly specifically configured in accordance with ISO CD 80369-3. Configuring one or more components of the present integrated valve assembly in accordance with this design specification permits efficient and convenient use of the valve assembly in enteral nutritional administration systems, while precluding inadvertent use of the valve assembly in other applications, such as for intravenous application.

In accordance with the illustrated embodiment, integrated valve assembly 10 comprises a first connector element 12, and a second connector element 14 which are joined to each other to form the integrated valve assembly. In accordance with the present invention, the first connector element 10 includes a first connection portion 16 at one thereof, and a first annular valve interface 18 in an opposite end thereof, with the connector element defining a first, internal flow passage 20.

In this embodiment, the first connection portion 16 of the first connector element 12 comprises a male threaded connector configured for enteral nutritional administration in accordance with ISO CD 80369-3. To this end, first connector element 12 includes an internally threaded, annular wall portion 22 configured in accordance with the above-noted ISO specification.

In this embodiment of the present integrated valve assembly, the second connector element 14 includes a second connection portion 24 at one end thereof, and a second annular valve interface 26 in an opposite end thereof. The second connector element 14 defines a second, internal flow passage 28, with the first and second connector elements 12 and 14 joined to each other with the first and second annular valve interfaces 18 and 26 arranged in confronting relationship with each other. As will be recognized by those familiar with the applicable ISO specification, the second connection portion 24 of second connector element 14 comprises a female threaded connector configured for enteral nutritional administration in accordance with ISO CD 80369-3. To this end, the second connection portion 24 includes an external thread formation 30 which is configured for threading engagement with an internal thread formation like that provided on annual wall portion 22.

In accordance with the present invention, a valve member is positioned between the first and second connector elements 12 and 14 at the first and second valve interfaces 18 and 26, with the valve member controlling flow between the first and second internal flow passages 20 and 28 of the first and second connector elements, thereby controlling flow through the valve assembly 10. In the preferred form, the valve member of the valve assembly is provided in the form of a resiliently deformable slit valve 40, which is positioned in captive relationship between the first and second connector elements 12 and 14.

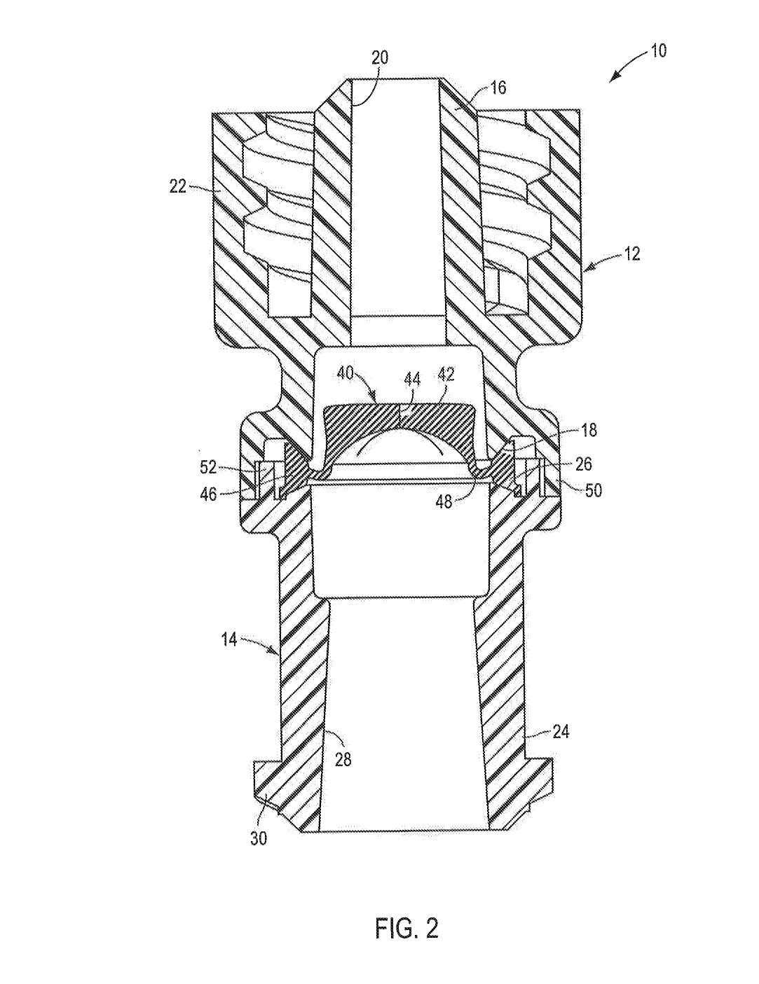

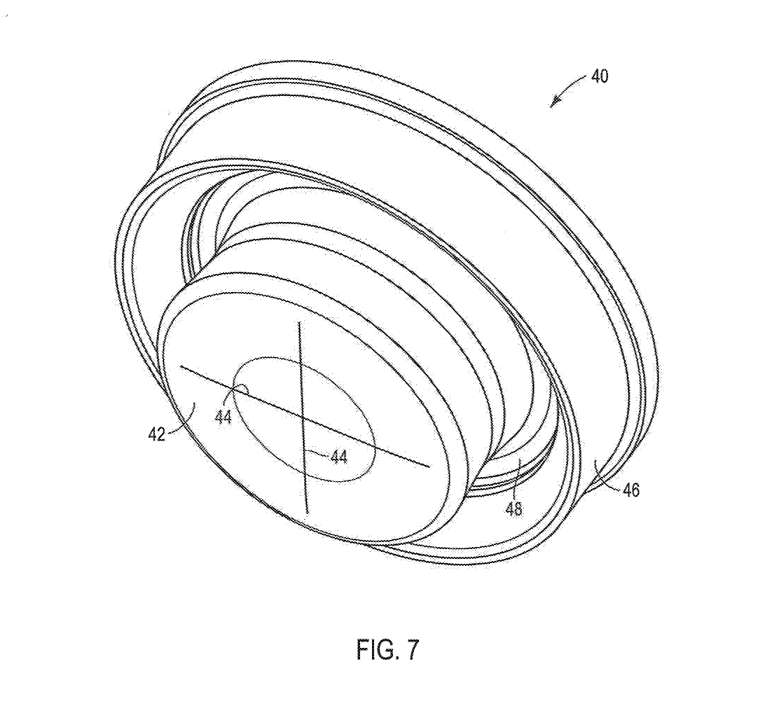

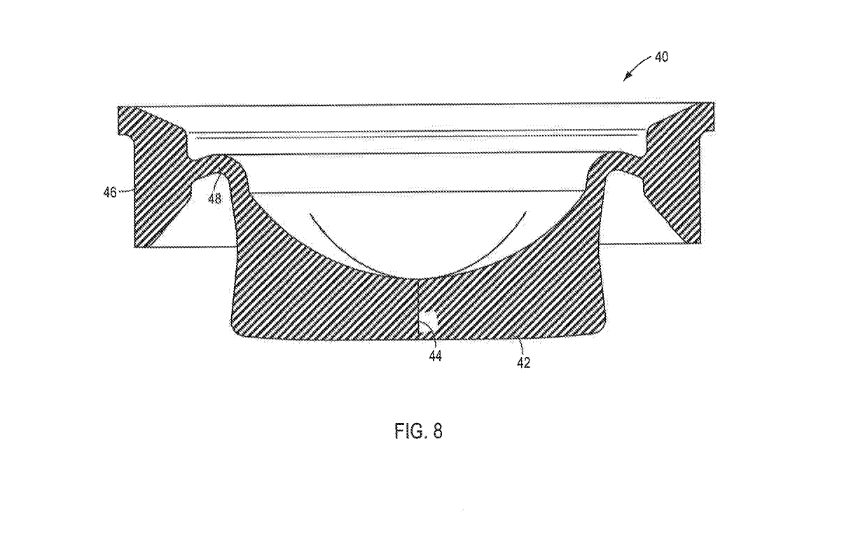

As shown in FIGS. 7 and 8, valve member 40 comprises a deformable head portion 42 defining at least one slit 44, with two such intersecting slits 44 shown in the illustrated embodiment. The valve member 40 further includes a peripheral attachment portion 46 which, as shown in FIG. 2, fits between the first annular valve interface 18 and second annular valve interface 26 of the valve assembly. The valve member 40 further includes an annular sleeve portion 48 which extends between and joins head portion 40 and peripheral attachment portion 46.

The specific configuration of valve member 40 can be varied while keeping with the principles disclosed herein for providing the desired pressure-responsive flow control. It is presently contemplated that the valve member may be configured in accordance with U.S. Pat. No. 5,839,614, the disclosure of which is hereby incorporated by reference. Other designs or types of valves (not illustrated) may be employed instead.

As noted, first connector element 12 and second connector element 14 are joined to each other with valve member 40 positioned between the connector elements at the first and second valve interfaces 18 and 26. To this end, the first connector element 12 includes a first annular attachment flange 50 positioned radially outwardly of first annular valve interface 18, while the second connector element 14 includes a second, annular attachment flange 52 positioned radially outwardly of second annular valve interface 26. During assembly of the integrated valve assembly 10, the valve member 40 is positioned between the first and second connector elements, with the peripheral attachment portion 46 positioned between the generally frusto-conical annular valve interfaces 18 and 26. The first and second connector portions are joined to each other such as by joining the annular attachment flanges 50, 52 to each other with suitable adhesive, sonic bonding, or the like.

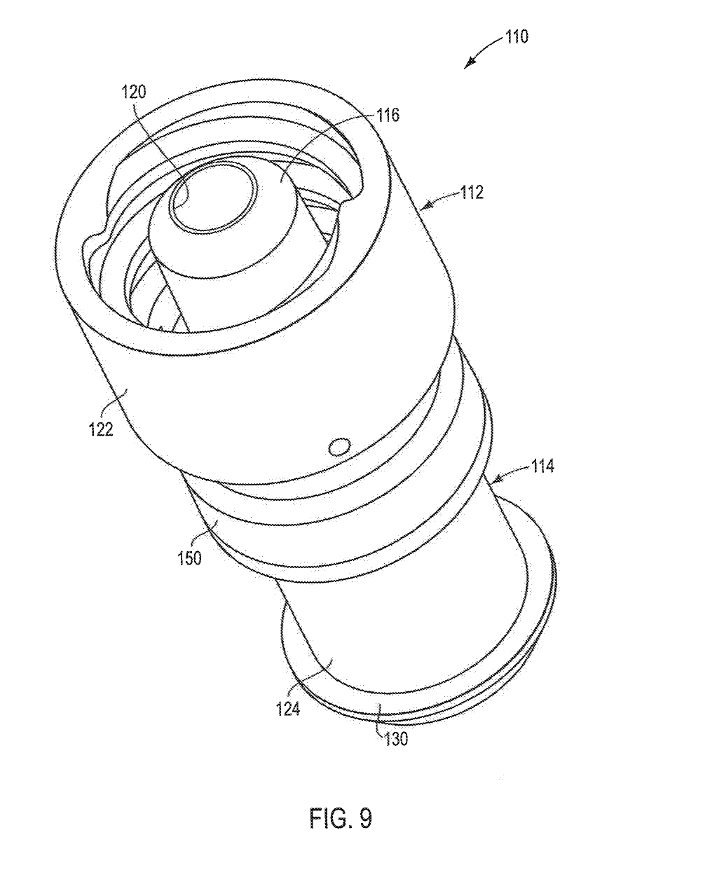

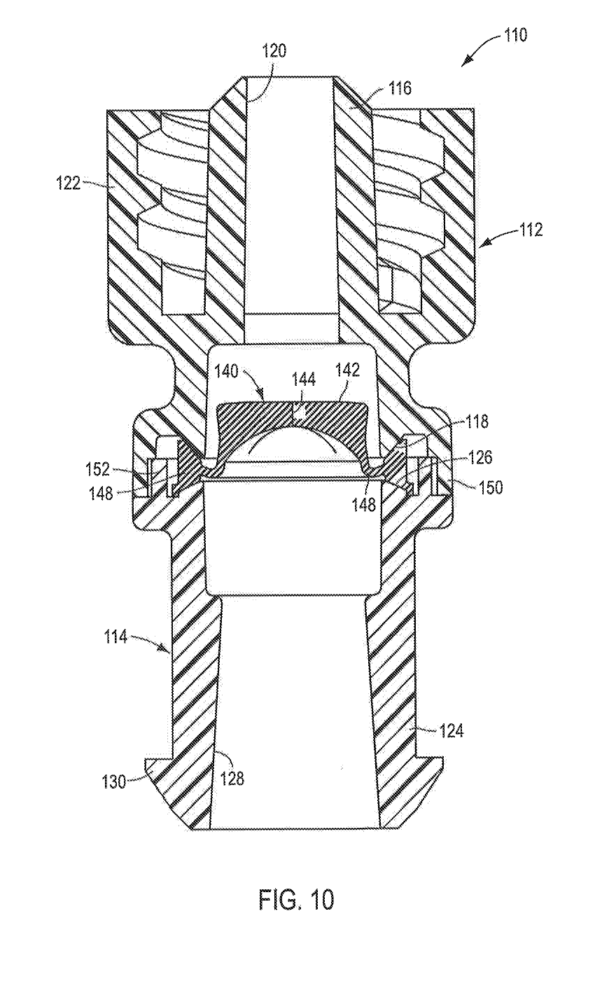

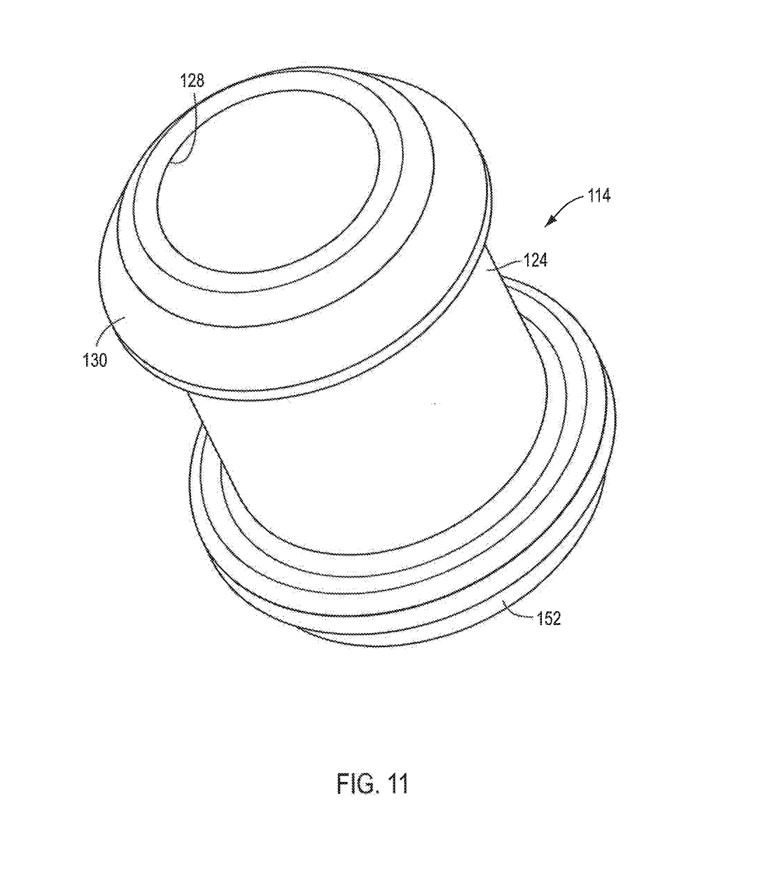

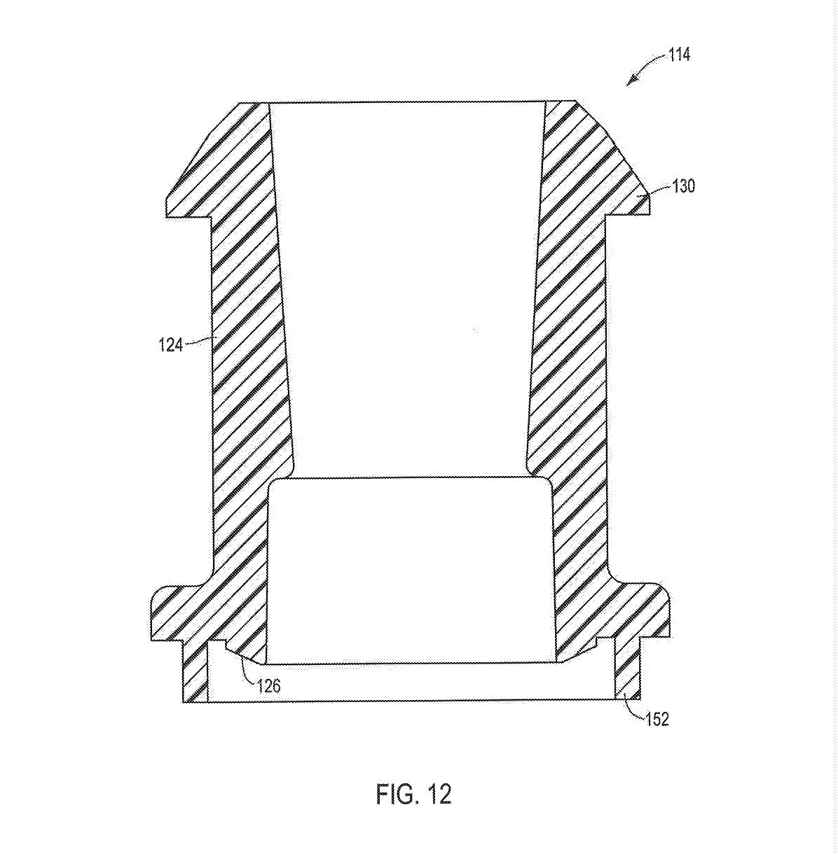

With reference now to FIGS. 9-12, therein is illustrated another preferred embodiment of the present integrated valve assembly, designated 110, with elements which generally correspond to those of the previously described embodiment designated by like reference numerals in the one-hundred series.

In this embodiment, integrated valve assembly 110 includes a first connector element 112, and a second connector element 114 which are joined to each other. The first connector element 112 includes a first connection portion 116 at one end therefore and a first annular valve interface 118 at an opposite end thereof, with the connector element defining a first, internal flow passage 120. In accordance with this embodiment of the present integrated valve assembly, the first connection portion 116 of the first connector element 112 comprises a male threaded connector configured for enteral nutritional administration in accordance with ISO CD 80369-3, and accordingly, includes an internally threaded annular wall portion 122. In this embodiment, the second connector element 114 includes a second connection portion 124 at one end thereof, and a second annular valve interface 126 at an opposite end therefore, with the connector element defining a second, internal flow passage 128. In accordance with this embodiment, the second connector element includes a second connection portion 124 comprising an annular undercut connector, and thus includes an annular connector element 130.

As in the previously described embodiment, integrated valve assembly 110 includes a valve member positioned between the first and second connector elements 112, 114, which valve member comprises a resiliently deformable slit valve 140, which may be configured in accordance with previously described valve member 40. To this end, the resiliently deformable slit valve 140 includes a head portion 142 defining at least one slit 144 (two being shown) a peripheral attachment portion positioned between the first annular valve interface 118 and the second, annular valve interface 126, and a sleeve portion 148 extending between and joining the head portion 142 and the peripheral attachment portion 146.

As in the previous embodiment, the resiliently deformable slit valve 140 is configured to permit flow in a first direction through the valve assembly 110 above a predetermined pressure differential acting in the first direction, and to prevent reverse flow in a second direction, opposite the first direction, through the valve assembly below a predetermined pressure differential acting in the second direction.

The resiliently deformable slit valve 40 is configured to permit flow in a first direction through the valve assembly 10 above a predetermined pressure differential acting in the first direction, and to prevent reverse flow in a second direction, opposite the first direction, through the valve assembly 10 below a predetermined pressure differential acting in the second direction.

The integrated valve assembly 110 is assembled by joining first connector element 112 to second connector element 114, with valve member 140 positioned therebetween. To this end, the first connector element 112 includes a first annular attachment flange positioned radially outwardly of the first annular valve interface 118, with the second connector element 114 including a second, annular attachment flange 152 positioned radially outwardly of second annular valve interface 126.

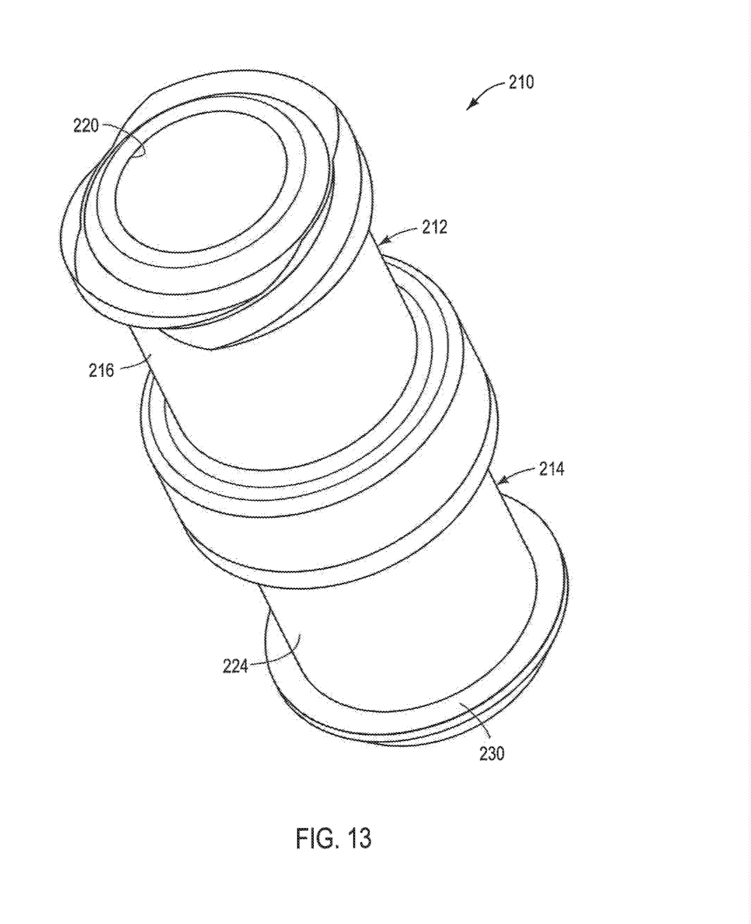

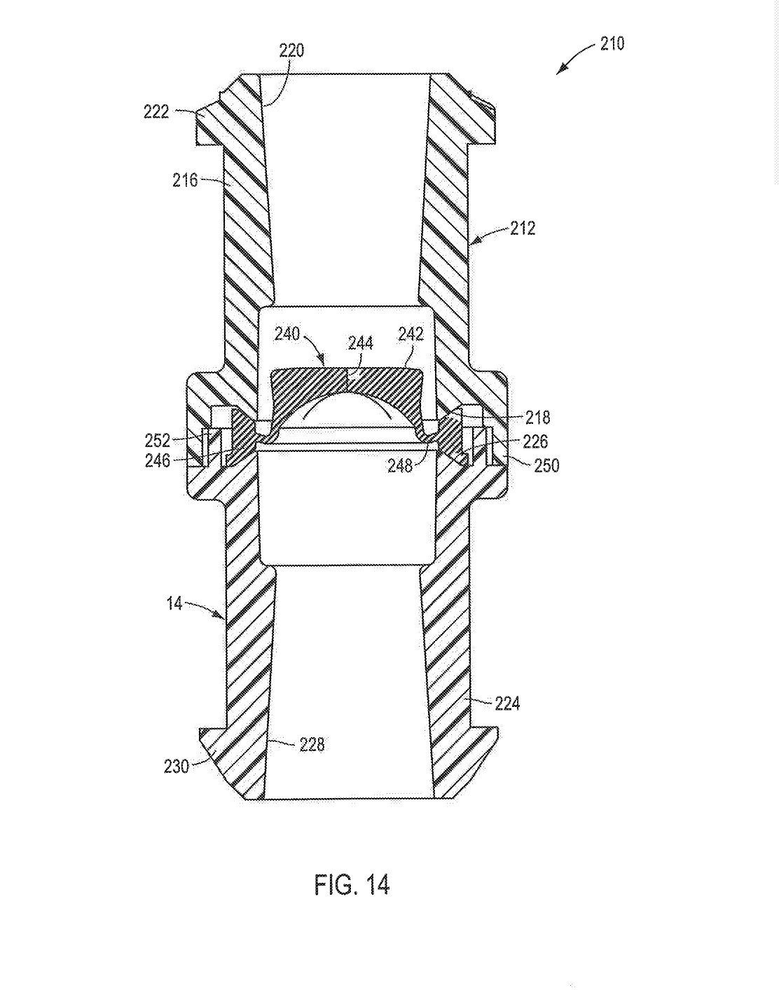

With reference now to FIGS. 13 and 14, therein is illustrated another preferred embodiment of the present integrated valve assembly, designated 210, with elements which generally correspond to those in the previously described embodiments designated by like reference numerals in the two-hundred series.

In this embodiment of the integrated valve assembly, designated 210, a first connector element 212 includes a first connection portion 216 at one end thereof, and a first annular valve interface 218, at an opposite end thereof, with the first connector element defining a first, internal flow passage 220. The second connector element 214 includes a second connection portion 224 at one end thereof, and a second annular valve interface 226 at an opposite end thereof, with the second connector element 214 defining a second, internal flow passage 228. In this illustrated embodiment, the first connector element 212 includes a first connection portion 216 which comprises a female threaded connector configured for enteral nutritional administration in accordance with ISO CD 80369-3, while the second connection portion 224 of the second connector element 214 comprises an annular undercut connector, including annular undercut element 230.

A valve member 240, comprising a resiliently deformable slit valve, is provided between the first and second connectors 212, 214, to permit flow in a first direction through the valve assembly 210 above a predetermined pressure differential acting in the first direction, and to prevent reverse flow in a second direction, opposite the first direction, to the valve assembly 210 below a predetermined pressure differential acting in the second direction.

As in the previous embodiment, the valve member 240 includes a head portion 242 defining at least one slit 244 (two being shown), a peripheral attachment portion 246 positioned between the first, annular valve interface 218 and second, annular valve interface 226, and a sleeve portion 248 extending between and joining the head portion 242 and the peripheral attachment portion 246. Valve member 240 can be configured in accordance with the previously-described embodiments.

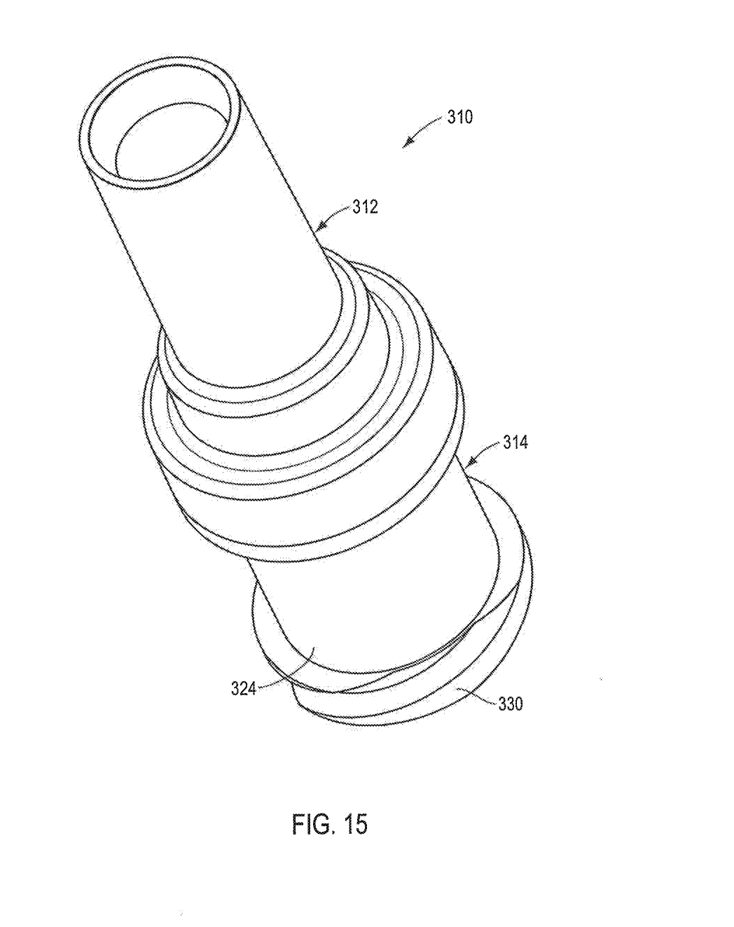

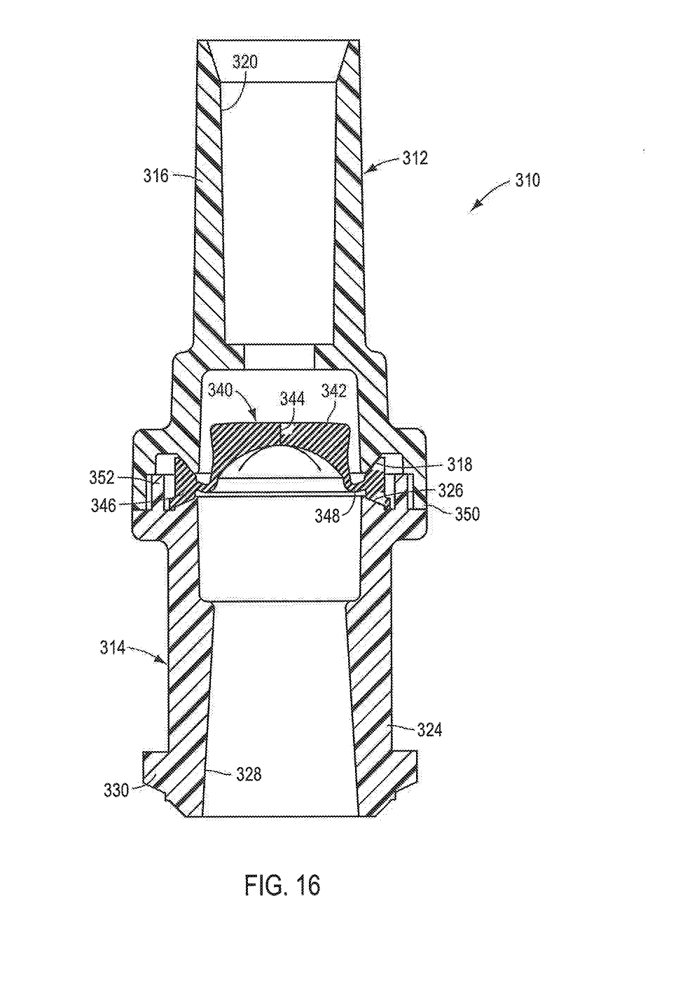

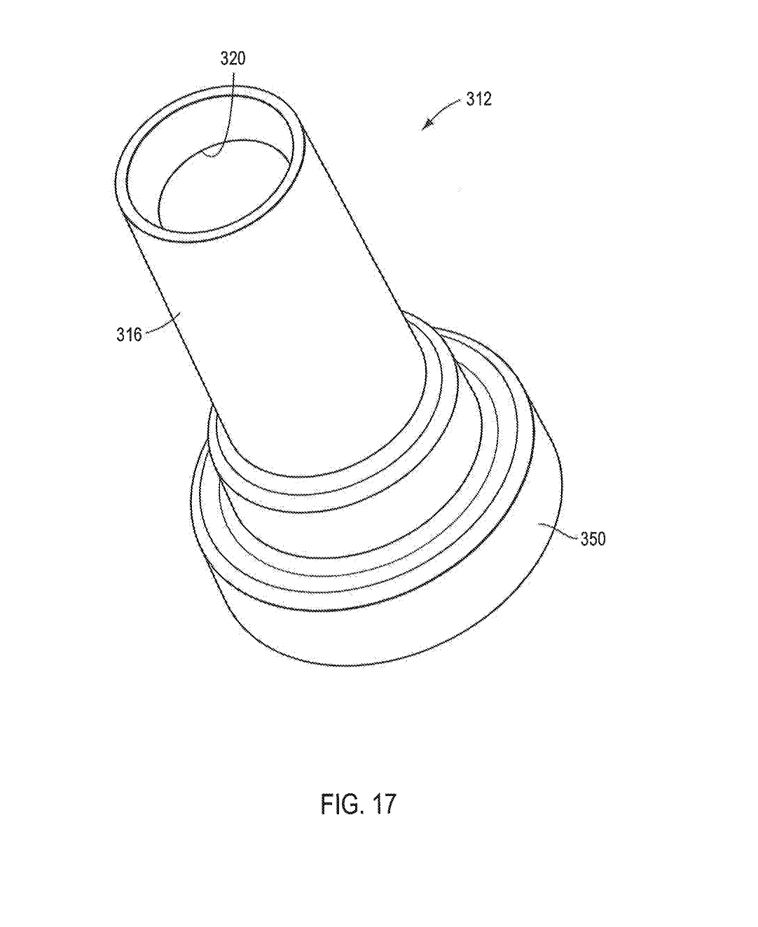

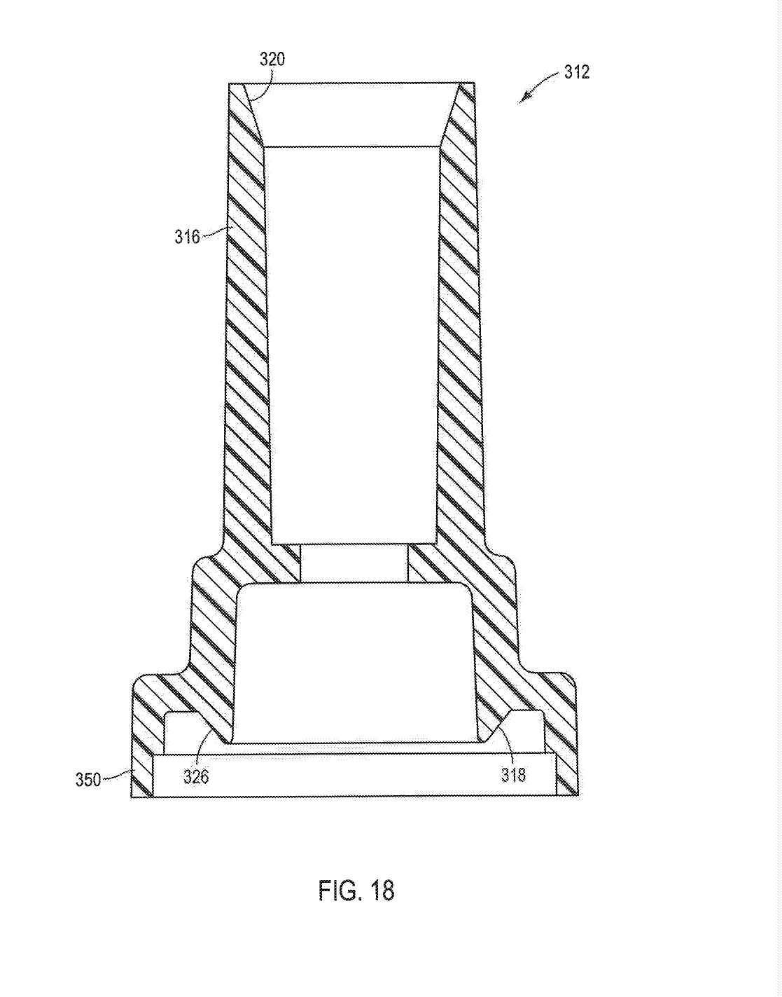

With reference now to FIGS. 15-18, therein is illustrated a further preferred embodiment of the present integrated valve assembly, designated 310, with elements which generally correspond to those of the previously-described embodiments designated by like reference numerals in the three-hundred series.

In accordance with the present invention, integrated valve assembly 310 includes a first connector element 312 and a second connector element 314. The first connector element includes a first connection portion 316 at one end thereof, and a first annular valve interface 318, at an opposite end thereof, with the first connector element defining a first, internal flow passage 320. In this embodiment, the first connection element 316 of the first connector element 312 comprises a tubular connector.

The second connector element 314 of the integrated valve assembly 310 includes a second connection portion 324 at one end thereof, and a second annular valve interface 326 at an opposite end thereof, with the second connector element defining a second, internal flow passage 328. In this embodiment, the second connection portion 324 of the second connector element 314 comprises a female threaded connector configured for enteral nutritional administration in accordance with ISO CD 80369-3.

As in the previous embodiments, a valve member, designated 340, is positioned between the first and second connector elements 312, 314, and controls flow between the first and second internal flow passages 320 and 328 for controlling flow through the valve assembly. The valve member 340 preferably comprises a resiliently deformable slit valve configured to permit flow in a first direction through the valve assembly 310 above a predetermined pressure differential acting in the first direction, and to prevent reverse flow in a second direction, opposite the first direction, through the valve assembly 310 below a predetermined pressure differential acting in the second direction.

As in the previous embodiments, the valve member 340 includes a head portion 342 defining at least one slit 344 (two being shown), a peripheral attachment portion 346 positioned between first annular valve interface 318 and second annular valve interface 326, and a sleeve portion 348 extending between and joining the head portion 342 and the peripheral attachment portion 346.

First connector element 312 is joined to second connector element 314, with valve member 340 positioned therebetween, by the provision of a first annular attachment flange 350 positioned radially outwardly of the first annular valve interface 318, and a second, annular attachment flange 352 positioned radially outwardly of second annular valve interface 326.

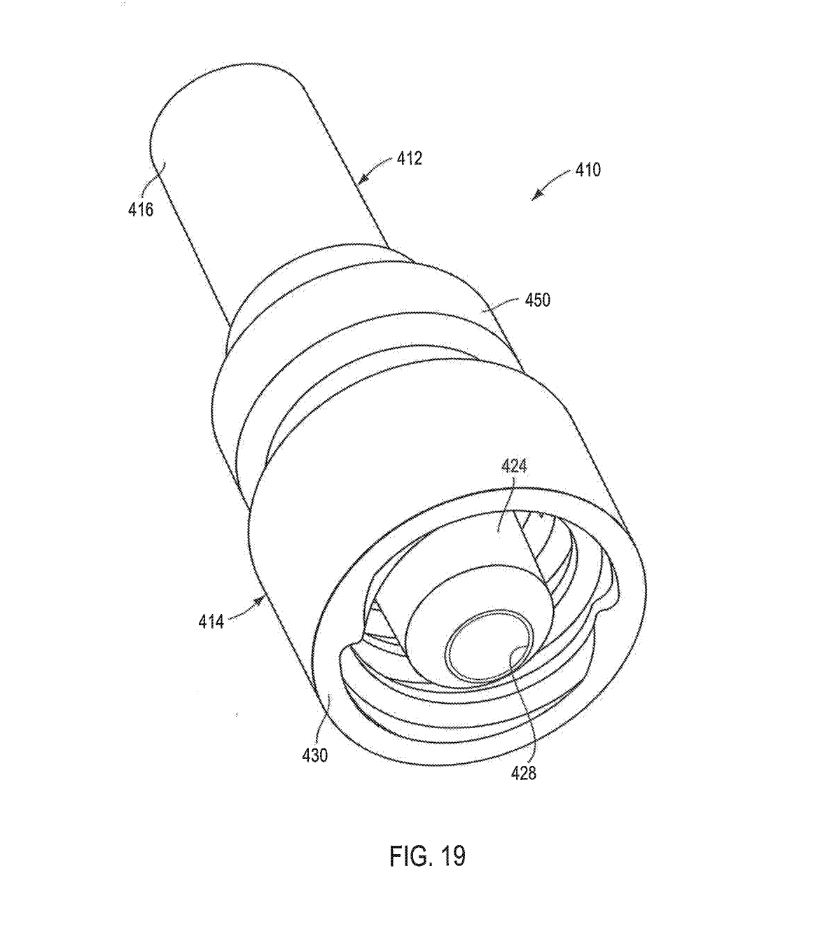

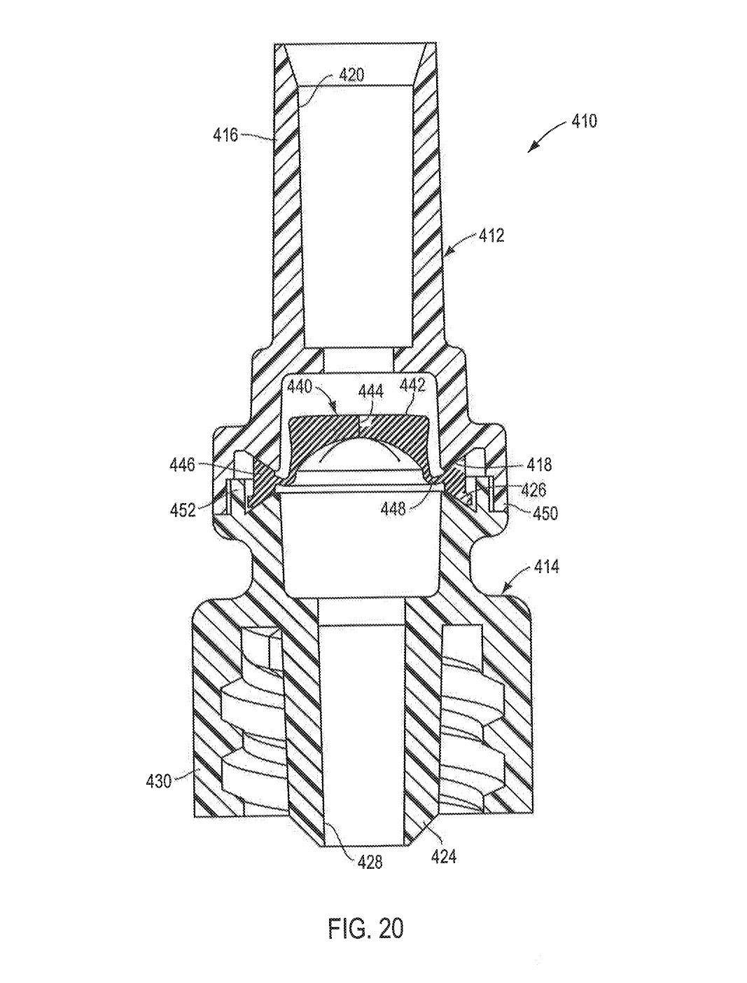

With reference now to FIGS. 19 and 20, therein is illustrated a further preferred embodiment of the present integrated valve assembly, designated 410. In this embodiment, elements which generally correspond to those elements of the previously described embodiments are designated by like reference numerals in the four-hundred series.

Integrated valve assembly 410 includes a first connector element 412, and a second connector element 414 which are joined to each other. The first connector element 412 has a first connection portion 416 at one end therefore, and a first annular valve interface 418 at an opposite end thereof. The first connector element 412 defines a first, internal flow passage 420.

The second connector element 414 has a second connection portion 424 at one end thereof, and a second annular valve interface 426, at an opposite end thereof, with the second connector element defining a second, internal flow passage. The first and second connector elements 412 and 414 are joined to each other with the first and second valve interfaces arranged in confronting relationship with each other.

In this embodiment, the first connection portion 416 of the first connector element 412 comprises a tubular connector, while the second connection portion 424 of the second connector element 414 comprises a male threaded connector configured for enteral nutritional administration in accordance with ISO CD 80369-3. To this end, the second connector element 414 includes an internally threaded annular wall 430 for providing the desired connection with components matching the above-referenced ISO specification.

The valve member 440 preferably comprises a resiliently deformable slit valve, comprising a head portion 442 defining at least one slit 444 (two being shown), a peripheral attachment portion 446 positioned between first, annular valve interface 418, and second annular valve interface 426, and a sleeve portion 448 extending between and joining the head portion and the peripheral attachment portion. The valve member is configured to permit flow in a first direction through the valve assembly 410 above a predetermined pressure differential acting in the first direction, and to prevent reverse flow in a second direction, opposite the first direction, through the valve assembly 410 below a predetermined pressure differential acting in the second direction.

The first connector element 412 and the second connector element 414 are joined to each other by the provision of a first annular attachment flange 450 positioned radially outwardly of first annular valve interface 418, and a second annular attachment flange 452 positioned radially outwardly of second annular valve interface 426.

The present invention can be summarized in the following statements or aspects numbered 1-12:

1. An integrated valve assembly (10, 110, 210, 310, 410), comprising:

a first connector element (12, 112, 212, 312, 412) having a first connection portion (16, 116, 216, 316, 416) at one end thereof, and a first annular valve interface at an opposite end thereof, said first connector element (12, 112, 212, 312, 412) defining a first, internal flow passage (20, 120, 210, 310, 410);

a second connector element (14,114, 214, 314, 414) having a second connection portion (24, 124, 224, 324, 424) at one end thereof, and an second annular valve interface at an opposite end thereof, said second connector element (14,114, 214, 314, 414) defining a second, internal flow passage (28, 128, 228, 328, 428), said first and second connector elements be joined to each other with said first and second valve interfaces arranged in confronting relationship with each other; and

a valve member (40, 140, 240, 340, 440) positioned between said first and second connector elements at said first and second valve interfaces, said valve member controlling flow between said first internal flow passage (20, 120, 220, 320, 420) and said second internal flow passage (28, 128, 228, 328, 428) of said first and second connector elements for controlling flow through said valve assembly (10, 110, 210, 310, 410).

2. An integrated valve assembly (10, 110, 210, 310, 410) in accordance with aspect 1, wherein

at least one of said first connection portion (16,116, 216, 316, 416) of said first connector element (12, 112, 212, 312, 412) and said second connection portion (24, 124, 224, 324, 424) of said second connector element (14,114, 214, 314, 414) being configured for nutritional administration in accordance with ISO CD 80369-3.

3. An integrated valve assembly (10, 110, 210, 310, 410) in accordance with aspect 1 or 2, wherein

said valve member (40, 140, 240, 320, 420) is configured to prevent flow in a first direction through said valve assembly (10, 110, 210, 310, 410) below a predetermined pressure differential.

4. An integrated valve assembly (10, 110, 210, 310, 410) in accordance with any of the preceding aspects, wherein

said valve member (40, 140, 240, 340, 440) is a resiliently deformable slit valve configured to permit flow in a first direction through said valve assembly (10, 110, 210, 310, 410) above a predetermined pressure differential acting in first direction, and to prevent reverse flow in a second direction, opposite said first direction, through said valve assembly (10, 110, 210, 310, 410) below a predetermined pressure differential acting in the second direction.

5. An integrated valve assembly (10) in accordance with aspect 2, wherein

said first connection portion (16) of said first connector element (12) comprises a male threaded connector configured for enteral nutritional administration in accordance with ISO CD 80369-3, and said second connection portion (24) of said second connector element (14) comprises a female threaded connector configured for enteral nutritional administration in accordance with ISO CD 80369-3,

said valve member (40) comprising a resiliently deformable slit valve configured to permit flow in a first direction through said valve assembly (10) above a predetermined pressure differential acting in the first direction, and to prevent reverse flow in a second direction, opposite said first direction, through said valve assembly (10) below a predetermined pressure differential acting the second direction.

6. An integrated valve assembly (110) in accordance with aspect 2, wherein

said first connection portion (116) of said first connector element (112) comprises a male threaded connector configured for enteral nutritional administration in accordance with ISO CD 80369-3, and said second connector portion (124) of said second connector element (114) comprises an annular undercut connector, said valve member (140) comprising a resiliently deformable slit valve configured to permit flow in a first direction through said valve assembly (110) above a predetermined pressure differential acting in a first direction, and to prevent reverse flow in a second direction, opposite said first direction, through said valve assembly (110) below a predetermined pressure differential acting in the second direction.

7. An integrated valve assembly (210) in accordance with aspect 2, wherein

said first connection portion (216) of said first connector element (212) comprises a female threaded connector configured for enteral nutritional administration in accordance with ISO CD 80369-3, and said second connection portion (224) of said second connector element (214) comprises an annular undercut connector,

said valve member (240) comprising a resiliently deformable slit valve configured to permit flow in a first direction through said valve assembly (210) above a predetermined pressure differential acting a first direction, and to prevent reverse flow in a second direction, opposite said first direction, through said valve assembly (210) below a predetermined pressure differential in the second direction.

8. An integrated valve assembly (310) in accordance with aspect 2, wherein

said first connection portion (316) of said first connector element (312) comprises tubular connector, and said second connection portion (324) of said second connector element (314) comprises a female threaded connector configured for enteral nutritional administration in accordance with ISO CD 80369-3,

said valve member (340) comprising a resiliently deformable slit valve configured to permit flow in a first direction through said valve assembly (310) above a predetermined pressure differential acting in the first direction, and to prevent reverse flow in a second direction, opposite said first direction, through said valve assembly (310) below a predetermined pressure differential acting in the second direction.

9. An integrated valve assembly (410) in accordance with aspect 2, wherein

said first connection portion (416) of said first connector element (412) comprises tubular connector, and said second connection portion (424) of said second connector element (414) comprises a male threaded connector configured for enteral nutritional administration in accordance with ISO CD 80369-3,

said valve member (440) comprising a resiliently deformable slit valve configured to permit flow in a first direction through said valve assembly (410) above a predetermined pressure differential acting in a the first direction, and to prevent reverse flow in a second direction, opposite said first direction, through said valve assembly (410) below a predetermined pressure differential acting in the second direction.

10. An integrated valve assembly (10, 110, 210, 310, 410) in accordance with any of the preceding aspects, wherein

said first annular valve interface of said first connector element (12, 112, 212, 312, 412) includes a first, annular valve seat (18, 118, 218, 318, 418), and a first annular attachment flange (50, 150, 250, 350, 450) positioned radially outwardly of said first annular valve seat (18, 118, 218, 318, 418), and said second annular valve interface of said second connector element (14, 114, 214, 314, 414) includes a second, annular valve seat (26, 126, 226, 326, 426), and a second annular attachment flange (52, 152, 252, 352, 452) positioned radially outwardly of said second annular valve seat (26, 126, 226, 326, 426), wherein said first and second annular attachment flanges are joined to each other.

11. An integrated valve assembly (10, 110, 210, 310, 410) in accordance with aspect 10, wherein

said valve member (40, 140, 240, 340, 440) is a resiliently deformable slit valve comprising a head portion (42, 142, 242, 342, 442) defining at least one slit (44, 144, 244, 344, 444), a peripheral attachment portion (46, 146, 246, 346, 446) positioned between said first, annular valve seat (18, 118, 218, 318, 418), and said second, annular valve seat (26, 126, 226, 326, 426), and a sleeve portion (48, 148, 248, 348, 448) extending between and joining said head portion (42, 142, 242, 342, 442) and said peripheral attachment portion (46, 146, 246, 346, 446).

12. An integrated valve assembly (10, 100, 210, 310, 410), comprising:

a first connector element (12, 112, 212, 312, 412) having a first connection portion (16, 116, 216, 316, 416) at one end thereof, and a first annular valve interface at an opposite end thereof, said first connector element (12, 112, 212, 312, 412) defining a first, internal flow passage (20, 120, 220, 320, 420);

a second connector element (14,114, 214, 314, 414) having a second connection portion (24, 124, 224, 324, 424) at one end thereof, and an second annular valve interface at an opposite end thereof, said second connector element (14,114, 214, 314, 414) defining a second, internal flow passage (28,128, 228, 328, 428), said first and second connector elements be joined to each other with said first second valve interfaces arranged in confronting relationship with each other,

said first annular valve interface of said first connector element (12, 112, 212, 312, 412) including a first, annular valve seat (18, 118, 218, 318, 418), and a first annular attachment flange (50, 150, 250, 350, 450) positioned radially outwardly of said first annular valve seat (18, 118, 218, 318, 418), and said second annular valve interface of said second connector element (14,114, 214, 314, 414) including a second, annular valve seat (26, 126, 226, 326, 426), and a second annular attachment flange (52, 152, 252, 352, 452) positioned radially outwardly of said second annular valve seat (26, 126, 226, 326, 426), wherein said first and second annular attachment flanges are joined to each other,

wherein at least one of said first connection portion (16, 116, 216, 316, 416) of said first connector element (12, 112, 212, 312, 412) and said second connection portion (24, 124, 224, 324, 424) of said second connector element (14, 114, 214, 314, 414) is configured for enteral nutritional administration in accordance with ISO CD 80369-3; and

a valve member (40, 140, 240, 340, 440) positioned between said first and second connector elements at said first and second valve interfaces, said valve member (40, 140, 240, 340, 440) controlling flow between said first internal flow passage (20, 120, 220, 320, 420) and said second internal flow passage (28, 128, 228, 328, 428) of said first connector element (12, 112, 212, 312, 412) and said second connector element (14, 114, 214, 314, 414) for controlling flow through said valve assembly (10, 110, 210, 310, 410), said valve member (40, 140, 240, 340, 440) comprising a resiliently deformable slit valve configured to permit flow in a first direction through said valve assembly (10, 100, 210, 310, 410), above a predetermined pressure differential acting in a first direction, and to prevent reverse flow in a second direction, opposite the first direction, through said valve assembly (10, 100, 210, 310, 410), below a predetermined pressure differential acting the second direction,

said valve member (40, 140, 240, 340, 440) comprising a head portion (42, 124, 242, 342, 442) defining at least one slit (44, 144, 244, 344, 444), a peripheral attachment portion (46, 146, 246, 346, 446) positioned between said first, annular valve seat (18, 118, 218, 318, 418) and said second, annular valve seat (26, 126, 226, 326, 426), and a sleeve portion (48, 148, 248, 348, 448) extending between and joining said head (42, 142, 242, 342, 442) portion and said peripheral attachment portion (46, 146, 246, 346, 446).

It will be readily observed from the foregoing detailed description of the invention and from the illustrations thereof that numerous other variations and modifications may be effected without departing from the true spirit and scope of the novel concepts or principles of this invention.

1. An integrated valve assembly (10, 110, 210, 310, 410), comprising:
a first connector element (12, 112, 212, 312, 412) having a first connection portion (15, 116, 216, 316, 416) at one end thereof, and a first annular valve interface at an opposite end thereof, said first connector element (12, 112, 212, 312, 412) defining a first, internal flow passage (20, 120, 210, 310, 410);
a second connector element (14,114, 214, 314, 414) having a second connection portion (24, 124, 224, 324, 424) at one end thereof, and an second annular valve interface at an opposite end thereof, said second connector element (14,114, 214, 314, 414) defining a second, internal flow passage (28, 128, 228, 328, 428), said first and second connector elements being joined to each other with said first and second valve interfaces arranged in confronting relationship with each other; and
a valve member (40, 140, 240, 340, 440) positioned between said first and second connector elements at said first and second valve interfaces, said valve member controlling flow between said first internal flow passage (20, 120, 220, 320, 420) and said second internal flow passage (28, 128, 228, 328, 428) of said first and second connector elements for controlling flow through said valve assembly (10, 110, 210, 310, 410).

2. An integrated valve assembly (10, 110, 210, 310, 410) in accordance with claim 1, wherein
at least one of said first connection portion (16,116, 216, 316, 416) of said first connector element (12, 112, 212, 312, 412) and said second connection portion (24, 124, 224, 324, 424) of said second connector element (14,114, 214, 314, 414) being configured for nutritional administration in accordance with ISO CD 80369-3.

3. An integrated valve assembly (10, 110, 210, 310, 410) in accordance with claim 1, wherein
said valve member (40, 140, 240, 320, 420) is configured to prevent flow in a first direction through said valve assembly (10, 110, 210, 310, 410) below a predetermined pressure differential.

4. An integrated valve assembly (10, 110, 210, 310, 410) in accordance with claim 1, wherein
said valve member (40, 140, 240, 340, 440) is a resiliently deformable slit valve configured to permit flow in a first direction through said valve assembly (10, 110, 210, 310, 410) above a predetermined pressure differential acting in first direction, and to prevent reverse flow in a second direction, opposite said first direction, through said valve assembly (10, 110, 210, 310, 410) below a predetermined pressure differential acting in the second direction.

5. An integrated valve assembly (10) in accordance with claim 2, wherein
said first connection portion (16) of said first connector element (12) comprises a male threaded connector configured for enteral nutritional administration in accordance with ISO CD 80369-3, and said second connection portion (24) of said second connector element (14) comprises a female threaded connector configured for enteral nutritional administration in accordance with ISO CD 80369-3,
said valve member (40) comprising a resiliently deformable slit valve configured to permit flow in a first direction through said valve assembly (10) above a predetermined pressure differential acting in the first direction, and to prevent reverse flow in a second direction, opposite said first direction, through said valve assembly (10) below a predetermined pressure differential acting the second direction.

6. An integrated valve assembly (110) in accordance with claim 2, wherein
said first connection portion (116) of said first connector element (112) comprises a male threaded connector configured for enteral nutritional administration in accordance with ISO CD 80369-3, and said second connector portion (124) of said second connector element (114) comprises an annular undercut connector, said valve member (140) comprising a resiliently deformable slit valve configured to permit flow in a first direction through said valve assembly (110) above a predetermined pressure differential acting in a first direction, and to prevent reverse flow in a second direction, opposite said first direction, through said valve assembly (110) below a predetermined pressure differential acting in the second direction.

7. An integrated valve assembly (210) in accordance with claim 2, wherein
said first connection portion (216) of said first connector element (212) comprises a female threaded connector configured for enteral nutritional administration in accordance with ISO CD 80369-3, and said second connection portion (224) of said second connector element (214) comprises an annular undercut connector,
said valve member (240) comprising a resiliently deformable slit valve configured to permit flow in a first direction through said valve assembly (210) above a predetermined pressure differential acting a first direction, and to prevent reverse flow in a second direction, opposite said first direction, through said valve assembly (210) below a predetermined pressure differential in the second direction.

8. An integrated valve assembly (310) in accordance with claim 2, wherein
said first connection portion (316) of said first connector element (312) comprises tubular connector, and said second connection portion (324) of said second connector element (314) comprises a female threaded connector configured for enteral nutritional administration in accordance with ISO CD 80369-3,
said valve member (340) comprising a resiliently deformable slit valve configured to permit flow in a first direction through said valve assembly (310) above a predetermined pressure differential acting in the first direction, and to prevent reverse flow in a second direction, opposite said first direction, through said valve assembly (310) below a predetermined pressure differential acting in the second direction.

9. An integrated valve assembly (410) in accordance with claim 2, wherein
said first connection portion (416) of said first connector element (412) comprises tubular connector, and said second connection portion (424) of said second connector element (414) comprises a male threaded connector configured for enteral nutritional administration in accordance with ISO CD 80369-3,
said valve member (440) comprising a resiliently deformable slit valve configured to permit flow in a first direction through said valve assembly (410) above a predetermined pressure differential acting in a the first direction, and to prevent reverse flow in a second direction, opposite said first direction, through said valve assembly (410) below a predetermined pressure differential acting in the second direction.

10. An integrated valve assembly (10, 110, 210, 310, 410) in accordance with claim 1, wherein
said first annular valve interface of said first connector element (12, 112, 212, 312, 412) includes a first, annular valve seat (18, 118, 218, 318, 418), and a first annular attachment flange (50, 150, 250, 350, 450) positioned radially outwardly of said first annular valve seat (18, 118, 218, 318, 418), and said second annular valve interface of said second connector element (14, 114, 214, 314, 414) includes a second, annular valve seat (26, 126, 226, 326, 426), and a second annular attachment flange (52, 152, 252, 352, 452) positioned radially outwardly of said second annular valve seat (26, 126, 226, 326, 426), wherein said first and second annular attachment flanges are joined to each other.

11. An integrated valve assembly (10, 110, 210, 310, 410) in accordance with claim 10, wherein
said valve member (40, 140, 240, 340, 440) is a resiliently deformable slit valve comprising a head portion (42, 142, 242, 342, 442) defining at least one slit (44, 144, 244, 344, 444), a peripheral attachment portion (46, 146, 246, 346, 446) positioned between said first, annular valve seat (18, 118, 218, 318, 418), and said second, annular valve seat (26, 126, 226, 326, 426), and a sleeve portion (48, 148, 248, 348, 448) extending between and joining said head portion (42, 142, 242, 342, 442) and said peripheral attachment portion (46, 146, 246, 346, 446).

12. An integrated valve assembly (10, 100, 21.0, 310, 410), comprising:
a first connector element (12, 112, 212, 312, 412) having a first connection portion (16, 116, 216, 316, 416) at one end thereof, and a first annular valve interface at an opposite end thereof, said first connector element (12, 112, 212, 312, 412) defining a first, internal flow passage (20, 120, 220, 320, 420);
a second connector element (14,114, 214, 314, 414) having a second connection portion (24, 124, 224, 324, 424) at one end thereof, and an second annular valve interface at an opposite end thereof, said second connector element (14,114, 214, 314, 414) defining a second, internal flow passage (28,128, 228, 328, 428), said first and second connector elements being joined to each other with said first second valve interfaces arranged in confronting relationship with each other,
said first annular valve interface of said first connector element (12, 112, 212, 312, 412) including a first, annular valve seat (18, 118, 218, 318, 418), and a first annular attachment flange (50, 150, 250, 350, 450) positioned radially outwardly of said first annular valve seat (18, 118, 218, 318, 418), and said second annular valve interface of said second connector element (14,114, 214, 314, 414) including a second, annular valve seat (26, 126, 226, 326, 426), and a second annular attachment flange (52, 152, 252, 352, 452) positioned radially outwardly of said second annular valve seat (26, 126, 226, 326, 426), wherein said first and second annular attachment flanges are joined to each other,
wherein at least one of said first connection portion (16, 116, 216, 316, 416) of said first connector element (12, 112, 212, 312, 412) and said second connection portion (24, 124, 224, 324, 424) of said second connector element (14, 114, 214, 314, 414) is configured for enteral nutritional administration in accordance with ISO CD 80369-3; and
a valve member (40, 140, 240, 340, 440) positioned between said first and second connector elements at said first and second valve interfaces, said valve member (40, 140, 240, 340, 440) controlling flow between said first internal flow passage (20, 120, 220, 320, 420) and said second internal flow passage (28, 128, 228, 328, 428) of said first connector element (12, 112, 212, 312, 412) and said second connector element (14, 114, 214, 314, 414) for controlling flow through said valve assembly (10, 110, 210, 310, 410), said valve member (40, 140, 240, 340, 440) comprising a resiliently deformable slit valve configured to permit flow in a first direction through said valve assembly (10, 100, 210, 310, 410), above a predetermined pressure differential acting in a first direction, and to prevent reverse flow in a second direction, opposite the first direction, through said valve assembly (10, 100, 210, 310, 410), below a predetermined pressure differential acting the second direction,
said valve member (40, 140, 240, 340, 440) comprising a head portion (42, 124, 242, 342, 442) defining at least one slit (44, 144, 244, 344, 444), a peripheral attachment portion (46, 146, 246, 346, 446) positioned between said first, annular valve seat (18, 118, 218, 318, 418) and said second, annular valve seat (26, 126, 226, 326, 426), and a sleeve portion (48, 148, 248, 348, 448) extending between and joining said head (42, 142, 242, 342, 442) portion and said peripheral attachment portion (46, 146, 246, 346, 446).
